(19) United States
(12) Patent Application Publication
(10) Pub. No.: US 2014/0287179 A1
(43) Pub. Date: Sep. 25, 2014

(54) ELASTOMER MOLDED BODY FOR MEDICAL INSTRUMENT

(71) Applicant: OLYMPUS CORPORATION, Tokyo (JP)

(72) Inventors: HIROKAZU KAMIOKA, Tokyo (JP); Akinobu Nakano, Tokyo (JP)

(21) Appl. No.: 14/299,242

(22) Filed: Jun. 9, 2014

Related U.S. Application Data
(63) Continuation of application No. PCT/JP2013/060463, filed on Apr. 5, 2013.

(30) Foreign Application Priority Data
Apr. 20, 2012 (JP) 2012-096582

(51) Int. Cl. A61L 31/04

(52) U.S. Cl. CPC A61L 31/041
USPC 428/36.92; 525/458; 525/453; 524/500

(57) ABSTRACT

An elastomer molded body for a medical instrument includes an elastomer material containing at least one kind of non-olefin-based elastomer, in which a melting point of the elastomer molded body is higher than a glass transition point of the elastomer molded body and the glass transition point of the elastomer molded body is in the range of 40° C. or more to 80° C. or less.

### CROSS REFERENCE TO RELATED APPLICATIONS

The present application is based on and claims priority under Japanese Patent Application No. 2012-096582 filed Apr. 20, 2012, and is a continuation application based on PCT/JP2013/060463 filed Apr. 5, 2013. The content of the Japanese Patent Application and the PCT applications are incorporated herein by reference.

### BACKGROUND OF THE INVENTION

1. Field of the Invention

The present invention relates to an elastomer molded body for a medical instrument.

2. Description of Related Art

An elastomer molded body having flexibility is used for most members constituting medical instruments such as an endoscope and a catheter. A soft elastomer is molded and then used as the elastomer molded body in many cases. As the soft elastomer, a non-olefin-based elastomer such as a thermoplastic polyurethane-based elastomer or a polyamide-based elastomer is used in terms of material properties required in the use of medical instruments, for example, tear strength, tensile strength, and thermal adhesiveness.

Examples of improving smoothness of releasing the molded body from a mold include a method of blending a releasing agent and a method of providing a resin layer with excellent releasing properties on the surface. For example, Japanese Patent No. 2886114 discloses a composite molded body using a styrene-based thermoplastic elastomer layer whose releasing properties are improved by blending a paraffin-based oil or the like, as a surface layer material. Accordingly, it is necessary to improve the releasing properties without damaging the characteristics of the non-olefin-based elastomer itself.

### SUMMARY OF THE INVENTION

According to a first aspect of the present invention, an elastomer molded body for a medical instrument, includes an elastomer material containing at least one kind of non-olefin-based elastomer, wherein a melting point of the elastomer molded body is higher than a glass transition point of the elastomer molded body and the glass transition point of the elastomer molded body is in the range of 40° C. or more to 80° C. or less. According to a second aspect of the present invention in the above-described first aspect, the non-olefin-based elastomer may be thermoplastic.

According to a third aspect of the present invention in the above-described first or second aspect, the non-olefin-based elastomer may contain a urethane-based elastomer.

According to a fourth aspect of the present invention in any one of the above described first to third aspects, an inorganic filler may be contained in the elastomer molded body.

According to a fifth aspect of the present invention in any one of the above-described first to fourth aspects, the elastomer molded body may be formed in a cylindrical shape.

According to a sixth aspect of the present invention in any one of the above-described first to fifth aspects, the elastomer molded body may be used for an outer surface of a bending portion of an endoscope.

According to a seventh aspect of the present invention, the durometer hardness measured in conformity with JIS K6253 may be 95 A or less, the tensile strength measured in conformity with JIS K6251 may be 12 Pa or more, and the tear strength measured in conformity with JIS K6252 may be 40 kN/m or more.

### DETAILED DESCRIPTION OF THE INVENTION

Hereinafter, the present invention will be described in detail.

A soft non-olefin-based elastomer used for an elastomer molded body for a medical instrument in the related art has an excessively low glass transition point of approximately −10 to −50° C. Therefore, the surface temperature (equivalent to the temperature of a molding die, generally room temperature) of the molded body at the time of release becomes considerably higher than the glass transition point. After further research had been performed by the present inventors based on this knowledge, the present inventors found that the releasing properties can be improved while the material characteristics are maintained by setting the glass transition point of the non-olefin-based elastomer itself constituting the elastomer molded body to be within a specific range.

An elastomer molded body for a medical instrument (hereinafter, simply referred to as a “molded body”) according to an embodiment of the present invention includes an elastomer material containing at least one kind of non-olefin-based elastomer, has a melting point higher than the glass transition point, and has a glass transition point (hereinafter, T_{g}) in the range of 40° C. or more to 80° C. or less and preferably in the range of 45° C. or more to 75° C. or less.

In the case where T_{g} of the molded body is 40° C. or higher, the temperature (generally, room temperature) of a molding die at the time of release becomes sufficiently lower than T_{g}. Consequently, the surface of the molded body is not sticky, the releasing properties are excellent, and the appearance of the molded body becomes better. Further, in the case where T_{g} is 80° C. or lower, the physical properties required for the use for medical instruments, for example, thermoplastic properties, tear strength, tensile strength, and thermal adhesiveness become sufficiently excellent.

Further, according to an embodiment of the present invention, when the non-olefin-based elastomer itself constituting an elastomer molding body has T_{g} in the range of 40° C. or more to 80° C. or less, sufficient releasing properties can be obtained even when the elastomer molded body is formed of only the non-olefin-based elastomer. Therefore, it is not necessary to blend components other than the non-olefin-based elastomer for improving the releasing properties, and the material characteristics are prevented from degrading due to blending the components.

In the case where a molded body has a plurality of T_{g}s, at least one may be in the range of 40° C. or more to 80° C. or less and a T_{g} may be out of the range of 40 to 80° C.

T_{g} of the molded body or the non-olefin-based elastomer can be measured by a differential scanning calorimeter (DSC).

In the case where the molded body is formed from a plurality of components, T_{g} of a kneaded material obtained by kneading each component at a temperature (a temperature equal to or higher than the highest melting temperature when a plurality of non-olefin-based elastomers are included) equal to or higher than the melting point of contained non-olefin-based elastomer is measured.

As the non-olefin-based elastomer constituting the elastomer material, rubber (thermosetting elastomer) or a thermoplastic elastomer is exemplified.

The non-olefin-based rubber and the thermoplastic elastomer respectively include polyurethane-based, polyester-based, polyamide-based, acrylic-based, and silicone-based, and the like.

These non-olefin-based elastomers may be used alone or in a combination of two or more kinds thereof.

As these non-olefin-based elastomers, elastomers synthesized by a known synthesizing method or commercially available elastomers may be used.

As the non-olefin-based elastomer, among the above-described elastomers, the thermoplastic elastomer is preferable in terms of improvement in molding processability when a molded body is molded, and at least one kind selected from polyurethane-based, polyester-based, polyamide-based, acrylic-based, and silicone-based is more preferable.

Particularly, as the non-olefin-based elastomer, it is preferable at least to contain a urethane-based elastomer from viewpoints that tear strength, abrasion resistance, and the like are excellent, a great number of products are commercially available, and there are various kinds of T_{g}.

In the case where an elastomer material contains one kind of non-olefin-based elastomer, a non-olefin-based elastomer having T_{g} in the range of 40° C. or more to 80° C. or less is used.

In the case where an elastomer material contains two or more kinds of non-olefin-based elastomers, a kneaded material obtained by melting and kneading those non-olefin-based elastomers may have T_{g} in the range of 40° C. or more to 80° C. or less. A non-olefin-based elastomer which does not have T_{g} in the range of 40° C. or more to 80° C. or less when it is used alone, may be contained.

In the case where an elastomer material contains a combination of two or more kinds of non-olefin-based elastomers having different T_{g}, that is, in the case where an elastomer material is a kneaded material of two or more kinds of non-olefin-based elastomers having different T_{g}, the following (1) to (3) can be exemplified as a combination of two or more kinds of non-olefin-based elastomer:

(1) A combination of two or more kinds of non-olefin-based elastomers having T_{g} in the range of 40° C. or more to 80° C. or less;

(2) A combination of at least one kind of non-olefin-based elastomer having T_{g} in the range of 40° C. or more to 80° C. or less and at least one kind of non-olefin-based elastomer which does not have T_{g} in the range of 40° C. or more to 80° C. or less; and

(3) A combination of two or more kinds of non-olefin-based elastomers which do not have T_{g} in the range of 40° C. or more to 80° C. or less.

Among (1) to (3) described above, preferable examples of (3) include a combination of a non-olefin-based elastomer having T_{g} lower than 40° C. and a non-olefin-based elastomer having T_{g} higher than 80° C. A kneaded material having T_{g} in the range of 40° C. or more to 80° C. or less can be obtained by melting and kneading these non-olefin-based elastomers. In addition, T_{g} can be finely adjusted by adjusting the blending ratio of respective non-olefin-based elastomers. There is an advantage that such a non-olefin-based elastomer having low T_{g} is easily obtained. T_{g} of most of soft elastomers which have been used for medical instruments is approximately −10 to −50° C. as described above. By mixing, a non-olefin-based elastomer with high T_{g} which is higher than 80° C., into them to adjust T_{g} to be in the range of 40 to 80° C., the releasing properties can be improved while material characteristics are ensured,

In terms of easily obtaining elastomer materials having a target T_{g}, it is preferable to combine two or more kinds of non-olefin-based elastomers having different T_{g} as the above-described (2) or (3).

When T_{g} is adjusted by combining two or more kinds of non-olefin-based elastomers, non-olefin-based elastomers having compatibility are used. From a viewpoint of compatibilization, non-olefin-based elastomers having the same kind of resin (for example, urethane-based and urethane-based) are preferably used. In the case where non-olefin-based elastomers have compatibility, non-olefin-based elastomers (for example, urethane-based and ester-based) having different kinds of resins may be combined.

The content of the non-olefin-based elastomer in the elastomer material is preferably 5% by mass or more and more preferably 10% by mass or more. When the content thereof is 5% by mass or more, the molded body has sufficient flexibility and excellent stretching properties. The upper limit may be 100% by mass, and is not particularly limited. The amount can be appropriately set in consideration of the balance with other components when other components are optionally blended in.

The elastomer material may contain components other than the non-olefin-based elastomer as long as the effects of the present invention are not damaged, if necessary.

For example, the elastomer material may contain a filler as a reinforcing material.

Examples of the filler include an inorganic filler and an organic filler.

Examples of the inorganic filler, which are not particularly limited, include inorganic fibers such as asbestos, glass fibers, alumina fibers, and rock wool; carbon black, silica, barium sulfate, titanium oxide, aluminum oxide, calcium carbonate, calcium silicate, magnesium silicate, and aluminum silicate.

Examples of the organic filler, which are not particularly limited, include organic fibers such as cotton, wool, silk, hemp, nylon fibers, aramid fibers, vinylon fibers, polyester fibers, rayon fibers, acetate fibers, phenol formaldehyde fibers, polyphenylene sulfide fibers, acrylic fibers, polyvinyl chloride fibers, polyvinylidene fibers, polyurethane fibers, and tetrafluoroethylene fibers; a polytetrafluoroethylene resin, a polyethylene resin, a polypropylene resin, a phenol resin, a polyimide resin, a melamine resin, and a silicone resin. These fillers may be used alone or in a combination of multiple kinds thereof.

Among the examples described above, inorganic fillers are preferable because of chemical resistance or heat resistance. Among the inorganic fillers, at least one kind selected from silica, barium sulfate, titanium oxide, aluminum oxide, calcium carbonate, calcium silicate, magnesium silicate, aluminum silicate, and the like is preferable.

When a filler is contained, the content thereof is preferably in the range of 0.05 parts by mass to 50 parts by mass and more preferably in the range of 0.5 parts by mass to 15 parts by mass with respect to 100 parts by mass of the non-olefin-based elastomer in the elastomer material. In the case where the content thereof is 0.05 parts by mass or more, sufficient reinforcing effects can be obtained. In the case where the content thereof is 50 parts by mass or less, the molded body can be prevented from being extremely hard.

The elastomer material may contain carbon as a colorant. When the elastomer material contains carbon, effects that, for example, the molded body has a predetermined hardness due to adjusting the blending amount and that the heat resistance of the molded body is improved can be obtained in addition to a coloring effect.

When carbon is blended in, the blending amount is preferably in the range of 0.05 to 50 parts by mass and more preferably in the range of 0.5 to 15 parts by mass with respect to 100 parts by mass of the non-olefin-based elastomer in the elastomer material. In the case where the content thereof is 0.05 parts by mass or more, the blending effect of carbon can be sufficiently obtained. In the case where the content thereof is 50 parts by mass or less, the molded body can be prevented from being extremely hard.

(Production Method)

The molded body according to the embodiment of the present invention can be produced by molding the above-described elastomer material. The molded body can be produced by a known method except that an elastomer material having specific T_{g} is used.

An example of the production method will be described with reference to an example of a case where multiple kinds of elastomers are used in combination.

Firstly, multiple kinds of non-olefin-based elastomers are melted and kneaded at a temperature equal to or higher than the melting point of the contained non-olefin-based elastomers (a temperature equal to or higher than the highest melting point when a plurality of non-olefin-based elastomers are contained) by a kneading machine such as a double screw roll, a kneader, or a Banbury mixer. At this time, optional components such as a filler and a reinforcing carbon may be added thereto as needed. The blending ratio of the multiple kinds of non-olefin-based elastomers is set according to T_{g} of each non-olefin-based elastomer such that the kneaded material to be obtained has T_{g} in the range of 40° C. or more to 80° C. or less.

In addition, the molded body of the present invention can be obtained by molding the obtained kneaded material to a desired shape. As the molding method, a known rubber molding method such as an injection molding or an extrusion molding can be used.

In the case of the extrusion molding, a target molded body can be obtained by filling a kneaded material into a molding die having a desired shape, hot pressing at a temperature higher than T_{g} of the kneaded material, and then cooling the molding die to a temperature equal to or less than T_{g} of the kneaded material to release the kneaded material. By setting the cooling temperature, that is, the surface temperature of the molded body at the time of release to T_{g} or less of the kneaded material,the stickiness of the surface is suppressed and the releasing properties are improved, and thus a molded body with excellent appearance can be obtained.

In terms of releasing properties, the cooling temperature, which may be set to a temperature equal to or lower than T_{g} of the kneaded material, is preferably (T_{g}−5)° C. or lower and more preferably (T_{g}−10)° C. or lower. The lower limit thereof is not particularly limited, but 0° or higher is preferable when the cost or the like is considered.

The molded body according to an embodiment of the present invention has excellent releasing properties and excellent material physical properties required in the use for medical instruments as described above.

In terms of usability in the use for medical instruments, it is preferable for the molded body according to an embodiment of the present invention to satisfy all of the physical properties of the following (1) to (3):

(1) The durometer hardness measured in conformity with JIS K6253 is 95 A or less.

(2) The tensile strength measured in conformity with JIS K6251 is 12 Pa or more.

(3) The tear strength measured in conformity with JIS K6252 is 40 kN/m or more.

The shapes of the molded body according to an embodiment of the present invention, which are not particularly limited, can be appropriately selected according to the use thereof, for example, a cylindrical shape, a sheet shape, a rod shape, a ring shape, and various block shapes.

The molded body according to an embodiment of the present invention may be used as a member constituting medical instruments. Examples of the medical instruments include an endoscope, a catheter, and a packing. Specific examples of the member using the molded body include a shell (outer surface) of a bending portion of an endoscope, a fold-proof member of an endoscope, a switch button or a shell covering the switch button of an endoscope, an O-ring used inside an endoscope, and a catheter for treatment tools.

### EXAMPLES

Hereinafter, the present invention will be described in detail with reference to Examples, but is not limited thereto. In the Examples described below, “parts” means parts by mass.

Raw materials, measurement and evaluation methods used in Examples and Comparative Examples are as follows.

[Raw Materials]

From among used raw materials, the kinds of resins, trade names, manufacturer's names, T_{g}s, and hardnesses of elastomers are listed in Table 1. The term “TPU” means a thermoplastic urethane-based elastomer. Measurement of T_{g} of the styrene-based elastomer which is an olefin-based elastomer was not performed.

As silica, “Mini-Seal #5” (manufactured by U.S. Silica Company) was used.

TABLE 1

| Kind of | | Manufacturer's | | |
|---|---|---|---|---|
| resin | Trade name | name | T_{g} | Hardness |
| TPU | DiARY MM9020 | SMP Technologies | 90° C. | 70D |
| | | Inc. | | |
| | Elastollan C60A | BASF | −50° C. | 60A |
| Ester- | Hytrel SB654 | DU | −10° C. | 65A |
| based | | PONT-TORAY | | |
| elastomer | | CO., LTD. | | |
| Styrene- | RABARON | Mitsubishi | — | 67A |
| based | PJ7300C | Chemical | | |
| elastomer | | Corporation | | |

[Measurement and Evaluation Method]

<Measurement of Glass Transition Temperature T_{g}>

The glass transition temperature was measured by a differential scanning calorimeter (DSC).

<Measurement of Hardness>

The durometer hardness was measured by a method in conformity with JIS K6253.

<Measurement of Tensile Strength>

The tensile strength was measured by carrying out a tensile test in conformity with JIS K6251.

<Measurement of Tear Strength>

The tear strength was measured by carrying out a tear test in conformity with JIS K6252.

<Evaluation of Releasing Properties>

The appearance of the obtained molded product was visually observed, and the releasing properties were evaluated by the following criteria.

(Evaluation Criteria of Releasing Properties)

O: The appearance was good without deformation or tearing, or the like.

X: The appearance was poor with deformation, tearing, and the like.

### Example 1

TPU (70 parts) whose T_{g} is 90° C. and TPU (30 parts) whose T_{g} is −50° C. were melted and kneaded at 220° C. using a double screw extrusion molding machine to obtain a pellet-shaped kneaded material. T_{g} of the kneaded material was measured.

The obtained kneaded material (pellet shape) was molded to a sheet shape with a thickness of 2 mm using injection molding. The hardness, the tensile strength, and the tear strength of the obtained molded product were measured. The results are listed in Table 2.

Further, the obtained kneaded material (pellet shape) was molded to a cylindrical shape with an inner diameter of 8 mm, a thickness of 0.8 mm, and a length of 150 mm using injection molding (molding temperature: 220° C., cooling temperature:)40° to obtain a molded product (shell of a bending portion for an endoscope). The appearance of the obtained molded product was observed and the releasing properties were evaluated.

The results are listed in Table 2.

### Examples 2 to 4 and Comparative Examples 2 and 3

Preparation of a kneaded material, measurement of T_{g}, production of a molded product, measurement of physical properties (hardness, tensile strength, and tear strength), and evaluation of releasing properties were performed in the same manner as those of Example 1 except that the blending compositions of a kneaded material were changed as listed in Table 2. The results are listed in Table 2.

### Comparative Examples 1 and 2

Preparation of a kneaded material, measurement of T_{g}, production of a molded product, and measurement of physical properties (hardness, tensile strength, and tear strength) were performed in the same manner as those of Example 1 except that the blending compositions of a kneaded material were changed as listed in Table 2. The results are listed in Table 2.

### Comparative Example 3

TPU (100 parts) whose T_{g} is −50° C. and a styrene-based elastomer (100 parts) were molded using two single-screw extruders such that the TPU and the styrene-based elastomer became two layers, and a composite molded product having a sheet shape with a thickness of 2 mm was obtained by carrying out coextrusion at 220° C. Measurement of the hardness of the styrene-based elastomer layer side was performed on the obtained composite molded product. Further, the tensile strength and the tear strength of the composite molded product were measured. The results are listed in Table 2.

In addition, TPU (100 parts) whose T_{g} is −50° C. and a styrene-based elastomer (100 parts) were extruded and molded at 220° using two single-screw extruders such that the TPU became the inner layer and the styrene-based elastomer became the outer layer, and then a composite molded product having a cylindrical shape with an inner diameter of 8 mm, a thickness of 0.8 mm, and a length of 150 mm was obtained. The appearance of the obtained composite molded product was visually observed and the releasing properties were evaluated. The results are listed in Table 2.

TABLE 2

| | Examples | | | | Comparative Examples | | |
|---|---|---|---|---|---|---|---|
| | 1 | 2 | 3 | 4 | 1 | 2 | 3 |
| TPU (T_{g} 90° C.) | 70 parts | 90 parts | 50 parts | 70 parts | — | 50 parts | — |
| TPU (T_{g} −50° C.) | 30 parts | 10 parts | — | 30 parts | 100 parts | 50 parts | Inner layer |
| | | | | | | | 100 parts |
| Ester-based | — | — | 50 parts | — | — | — | — |
| elastomer | | | | | | | |
| (T_{g} −10° C.) | | | | | | | |
| Styrene-based | — | — | — | — | — | — | Outer layer |
| elastomer | | | | | | | 100 parts |
| Silica | — | — | — | 10 parts | — | — | — |
| Hardness | 88A | 95A | 76A | 92A | 65A | 78A | 70A*¹ |
| Tensile strength | 15 | 18 | 15 | 17 | 15 | 14 | 12*² |
| (MPa) | | | | | | | |
| Tear strength | 45 | 50 | 42 | 47 | 42 | 42 | 28*² |
| (kN/m) | | | | | | | |
| T_{g} (° C.) | 49 | 75 | 62 | 50 | −50 | 21 | — |
| Releasing properties | ◯ | ◯ | ◯ | ◯ | X | X | ◯ |
| (cooling temperature | | | | | | | |
| 40° C.) | | | | | | | |

*¹The hardness of the styrene-based elastomer layer side of the composite molded product was measured.
*²Evaluation was performed on the composite molded product.

A soft elastomer generally and conventionally used for an elastomer molded body for a medical instrument was used for the molded product of Comparative Example 1, the physical properties (hardness, tensile strength, and tear strength) thereof were excellent but the releasing properties thereof were poor as shown in the above-described results.

The molded product of Comparative Example 2 whose T_{g} is 21° C. had poor releasing properties and the tensile strength thereof was more degraded when compared to Comparative Example 1.

The releasing properties of the molded product of Comparative Example 3 in which the outer layer of the styrene-based elastomer was laminated on the inner layer of TPU was improved, but the tensile strength and the tear strength, especially the tear strength, were greatly degraded when compared to those of Comparative Example 1. Further, when evaluation of the adhesion between layers was performed on the molded product having a sheet shape of Comparative Example 3 using a method in conformity with a T type separating test (JIS K6854-3), two layers were easily separated.

In contrast, the tensile strength and the tear strength of the molded products of Examples 1 to 4 showed the same values as those of Comparative Example 1 or values superior to those of Comparative Example 1, and the releasing properties thereof was also improved.

The entire components described in the above-mentioned embodiments, and various modified examples can be carried out by suitably changing or deleting the combination within the scope of the technical idea of the invention. While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An elastomer molded body for a medical instrument, comprising:
an elastomer material containing at least one kind of non-olefin-based elastomer, wherein
a melting point of the elastomer molded body is higher than a glass transition point of the elastomer molded body and the glass transition point of the elastomer molded body is in the range of 40° C. or more to 80° C. or less.

2. The elastomer molded body for a medical instrument according to claim 1, wherein
the non-olefin-based elastomer is thermoplastic.

3. The elastomer molded body for a medical instrument according to claim 1, wherein
the non-olefin-based elastomer contains a urethane-based elastomer.

4. The elastomer molded body for a medical instrument according to claim 1, wherein
an inorganic filler is contained in the elastomer molded body.

5. The elastomer molded body for a medical instrument according to claim 1, wherein
the elastomer molded body is formed in a cylindrical shape.

6. The elastomer molded body for a medical instrument according to claim 1, wherein
the elastomer molded body is used for an outer surface of a bending portion of an endoscope.

7. The elastomer molded body for a medical instrument according to claim 1, wherein
the durometer hardness measured in conformity with JIS K6253 is 95 A or less, the tensile strength measured in conformity with JIS K6251 is 12 Pa or more, and the tear strength measured in conformity with JIS K6252 is 40 kN/m or more.
